(19) United States
(12) Patent Application Publication
(10) Pub. No.: US 2018/0185623 A1
(43) Pub. Date: Jul. 5, 2018

(54) CLAMP FOR USE IN ASSEMBLY OF A MICROPROTRUSION DEVICE AND CORRESPONDING METHOD

(71) Applicant: NanoPass Technologies Ltd., Nes Ziona (IL)

(72) Inventors: Rotem LESHER, Kfar Saba (IL); Gal ADMATI, Kibbutz Dorot (IL); Rom EISENBERG, KFAR SABA (IL)

(21) Appl. No.: 15/396,833

(22) Filed: Jan. 3, 2017

(51) Int. Cl. A61M 37/00

(52) U.S. Cl. CPC A61M 37/0015; A61M 2207/10; A61M 2037/0053

(57) ABSTRACT

A clamp (10) for use in assembly of a microneedle device (100) has a flexible collet (14) projecting upwards from a base (12). The flexible collet (14) grips an adapter (106) of the microneedle device (100). A displaceable fixture (16), with two locating edges (18), (20), is displaceably mounted to base (12) so as to be displaceable between a raised position for positioning an adapter on the flexible collet and a lowered position in which locating edges (18), (20) abut surfaces of adapter (106) to achieve precise positioning of the adapter in two dimensions. An attachment surface (108) of the adapter (106) projects above locating edges (18), (20) of fixture (16) so as to be accessible for attachment of substrate (102).

### FIELD AND BACKGROUND OF THE INVENTION

The present invention relates to manufacture of microprotrusion devices and, in particular, it concerns a clamp for use in assembly of a microprotrusion device and corresponding methods.

It is known to provide microprotrusion devices, i.e., devices which include protrusions of height dimensions up to 1 millimeter, which may be solid or hollow microneedles or other types of protrusion, typically for penetrating into one or more layers of tissue of a biological barrier. A primary example of interest is a microneedle chip carrying one or more hollow microneedle used for delivery of fluids or withdrawal of fluids to or from a biological barrier.

Due to their small dimensions, arrangements of microprotrusions must be manufactured to high precision, and are typically formed integrally with an underlying substrate, typically of sub-millimeter thickness. Typical materials for manufacture of microprotrusions and their substrate include, but are not limited to, silicon, various biocompatible polymers, and metal sheet material. In order to turn these small microprotrusion components into a useful device, for example, a manually manipulated device, the substrate needs to be attached to an “adapter” of relatively large dimensions. Particularly in the case of hollow microneedles, the adapter also needs to provide a fluid flow path in sealed interconnection with an internal bore of the hollow microneedles.

Typical materials for manufacture of such adapters are various polymer materials, and most preferably, injection molded plastic adapters which can be mass produced cheaply and efficiently. Such adapters tend to have significant variation in design parameters due to post-molding shrinkage or deformation, which may lead to variations in dimensions that are comparable to, or in some cases larger than, the dimensions of the microprotrusions and/or substrate. Precise alignment of a small-dimension, high-precision component such as the microprotrusions component with a much larger, low-precision component, as well as performance of processing steps such as the accurate application of small quantities of adhesive in precise alignment with both components, present significant manufacturing challenges.

### SUMMARY OF THE INVENTION

The present invention is a clamp for use in assembly of a microprotrusion device and corresponding methods.

According to the teachings of an embodiment of the present invention there is provided, a clamp for use in assembly of a microprotrusion device in which a substrate with a least one microprotrusion is to be attached to an adapter having an elongated body terminating in an attachment surface to which the substrate is to be attached, the clamp comprising: (a) a base; (b) a flexible collet mounted on the base so as to project upwards from the base, the flexible collet being configured for repositionably gripping at least one feature of the adapter; and (c) a displaceable fixture having at least two locating edges, the fixture being displaceably mounted to the base so as to be displaceable between a raised position for positioning an adapter on the flexible collet and a lowered position in which locating edges abut surfaces of the adapter to achieve precise positioning of the adapter in two dimensions, and wherein in the lowered position, the attachment surface of the adapter projects above the locating edges of the fixture so as to be accessible for attachment of the substrate.

According to a further feature of an embodiment of the present invention, the flexible collet is an internal collet configured for gripping an internal bore of the adapter.

According to a further feature of an embodiment of the present invention, the flexible collet has an internal channel, and further comprising a vacuum channel passing through the base, the vacuum channel being in sealing interconnection with the flexible collet to allow application of suction via the internal channel of the flexible collet.

According to a further feature of an embodiment of the present invention, the displaceable fixture is mounted to the base via a hinge.

According to a further feature of an embodiment of the present invention, the locating edges are implemented as internal surfaces of an aperture passing through the displaceable fixture.

According to a further feature of an embodiment of the present invention, there is also provided a depressor assembly comprising a depressor element configured for engaging an upward-facing surface of the adapter and an actuator associated with the depressor element and configured to selectively lower the depressor element so as to press the adapter further into engagement with the flexible collet, thereby bringing the attachment surface of the adapter to a predefined level relative to the base.

According to a further feature of an embodiment of the present invention, there is also provided a spring arrangement deployed so as to bias the displaceable fixture towards the lowered position.

There is also provided according to the teachings of an embodiment of the present invention, a method for assembling a microprotrusion device comprising the steps of: (a) providing the aforementioned clamp; (b) with the fixture in the raised position, positioning an adapter on the flexible collet so as to be gripped by the flexible collet, the adapter having an elongated body terminating in an attachment surface; (c) displacing the fixture to the lowered position so that the locating edges abut surfaces of the adapter to achieve precise positioning of the adapter in two dimensions; and (d) attaching to the attachment surface a substrate with a least one microprotrusion projecting therefrom to as to form a microprotrusion device.

According to a further feature of an embodiment of the present invention, prior to the attaching, a depressor element is lowered into engagement with an upward-facing surface of the adapter so as to press the adapter further into engagement with the flexible collet, thereby bringing the attachment surface of the adapter to a predefined level relative to the base.

According to a further feature of an embodiment of the present invention, the flexible collet is an internal collet that grips an internal bore of the adapter.

According to a further feature of an embodiment of the present invention, the at least one microprotrusion comprises at least one hollow microneedle.

According to a further feature of an embodiment of the present invention, the at least one microprotrusion is implemented as a plurality of microprotrusions.

According to a further feature of an embodiment of the present invention, the substrate and the at least one microprotrusion are integrally formed from a single crystal of silicon.

According to a further feature of an embodiment of the present invention, the adapter is formed with a female luer connector.

### BRIEF DESCRIPTION OF THE DRAWINGS

The invention is herein described, by way of example only, with reference to the accompanying drawings, wherein:

FIG. 1 is an isometric view of a clamp constructed and operative according to the teachings of an embodiment of the present invention, prior to use;

FIG. 2 is an isometric view of the clamp of FIG. 1 shown with a displaceable fixture in a raised position, and after initial positioning of an adapter;

FIG. 3 is an isometric view similar to FIG. 2 after lowering of the displaceable fixture, additionally showing a depressor element;

FIG. 4 is a vertical cross-sectional view taken through the clamp and adapter of FIG. 3, additionally showing a microneedle chip attached to the adapter;

FIG. 5A is an isometric view of an assembled microneedle adapter of FIG. 4; and

FIG. 5B is an enlarged isometric view of a hollow microneedle component from the adapter of FIG. 5A according to one particularly preferred implementation.

### DESCRIPTION OF THE PREFERRED EMBODIMENTS

The present invention is a clamp for use in assembly of a microprotrusion device and corresponding methods.

The principles and operation of clamps and corresponding methods according to the present invention may be better understood with reference to the drawings and the accompanying description.

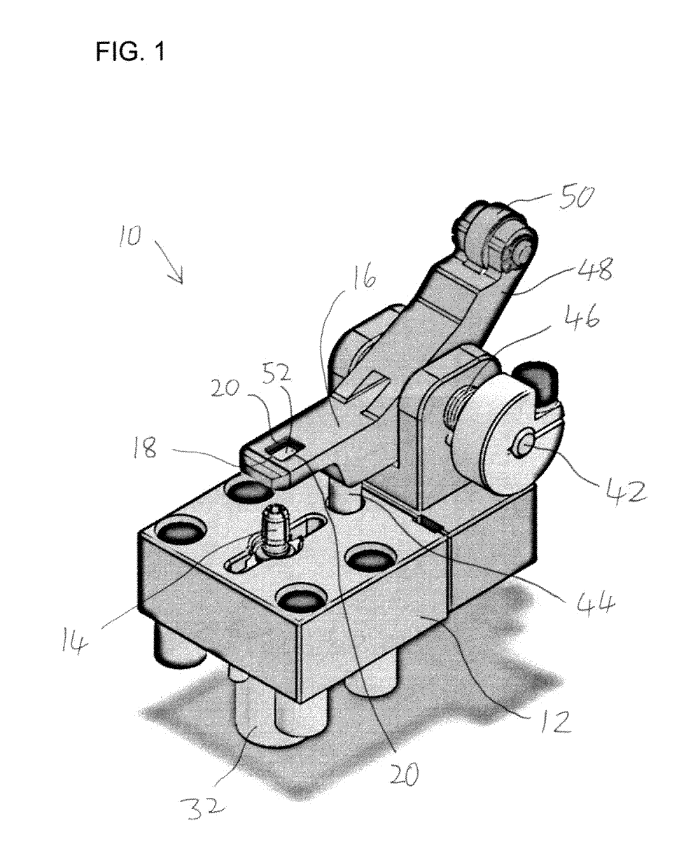

Referring now to the drawings, FIGS. 1-4 show various views and stages of use of a clamp, generally designated 10, constructed and operative according to an embodiment of the present invention, for use in assembly of a microprotrusion device 100 in which a substrate 102 with a least one microprotrusion 104 is to be attached to an adapter 106 having an elongated body terminating in an attachment surface 108 to which the substrate is to be attached.

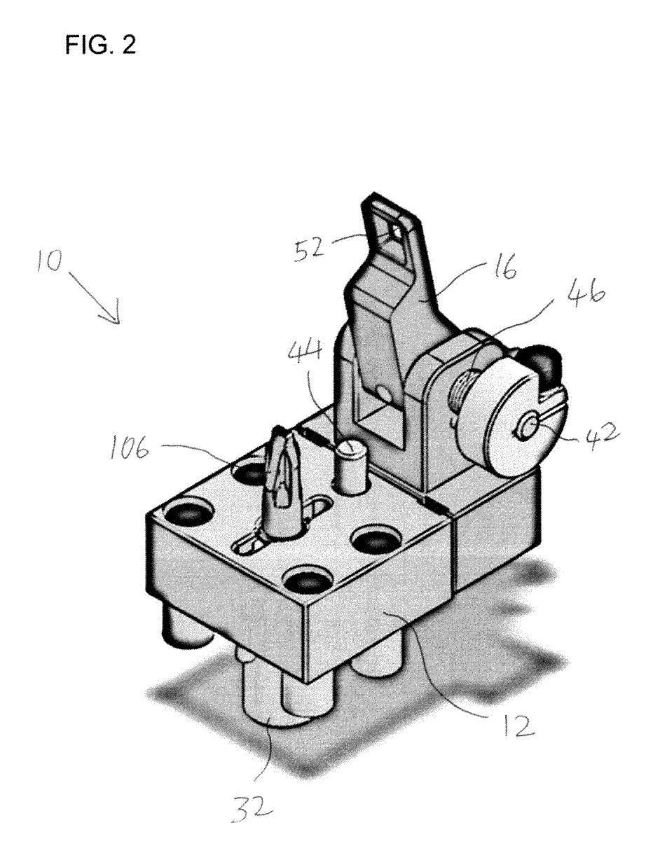

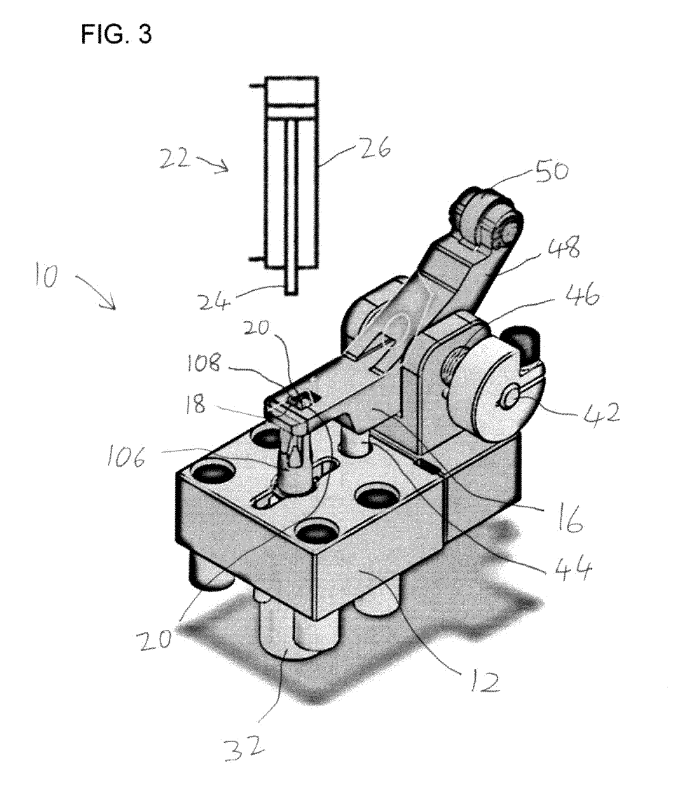

In general terms, clamp 10 includes a base 12 on which is mounted a flexible collet 14 so as to project upwards from the base. Flexible collet 14 is configured for repositionably gripping at least one feature of adapter 106. A displaceable fixture 16, having at least two locating edges 18, 20, is displaceably mounted to base 12 so as to be displaceable between a raised position (FIG. 2) for positioning an adapter on the flexible collet and a lowered position (FIG. 3) in which locating edges 18, 20 abut surfaces of adapter 106 to achieve precise positioning of the adapter in two dimensions. Displaceable fixture 16 is also configured so that, in the lowered position, the attachment surface 108 of adapter 106 projects above locating edges 18, 20 of fixture 16 so as to be accessible for attachment of substrate 102.

At this stage, it will readily be appreciated that an aspect of the present invention provides a solution for the aforementioned challenges relating to alignment of the attachment surface for attachment of substrate 102. Specifically, by using a flexible collet to grip adapter 106, the precise alignment of adapter can still be adjusted by bringing locating edges 18 and 20 into contact with corresponding surfaces of the adapter, all within the range of positions accommodated by the flexible collet. Particularly by employing alignment of surfaces of the adapter that are relatively close to attachment surface 108 (preferably in the upper third of the adapter closest to the attachment surface, and most preferably, within the upper fifth of the height nearest to attachment surface 108), it is possible to achieve precise alignment of the contact surface in a predefined position relative to clamp 10 to a level of precision which exceeds the overall manufacturing precision of the adapter as a whole, ready for precise alignment and attachment of a microprotrusion component thereon.

Most preferably, locating edges 18 and 20 achieve precise alignment of the attachment surface 108 in two horizontal (X-Y) dimensions, while a separate depressor assembly adjusts alignment of the surface in the vertical (Z) direction. An example of such a depressor assembly, designated 22, is shown schematically in FIG. 3 in which a depressor element 24 is configured for engaging an upward-facing surface of the adapter and an actuator 26, associated with the depressor element, is configured to selectively lower depressor element 24 so as to press adapter 106 further into engagement with flexible collet 14, thereby bringing attachment surface 108 of the adapter to a predefined level relative to base 12. Here too, the gripping of adapter 106 by flexible collet 14 facilitates the adjustment of the vertical alignment, and contributes to maintaining that alignment once depressor element 24 is retracted.

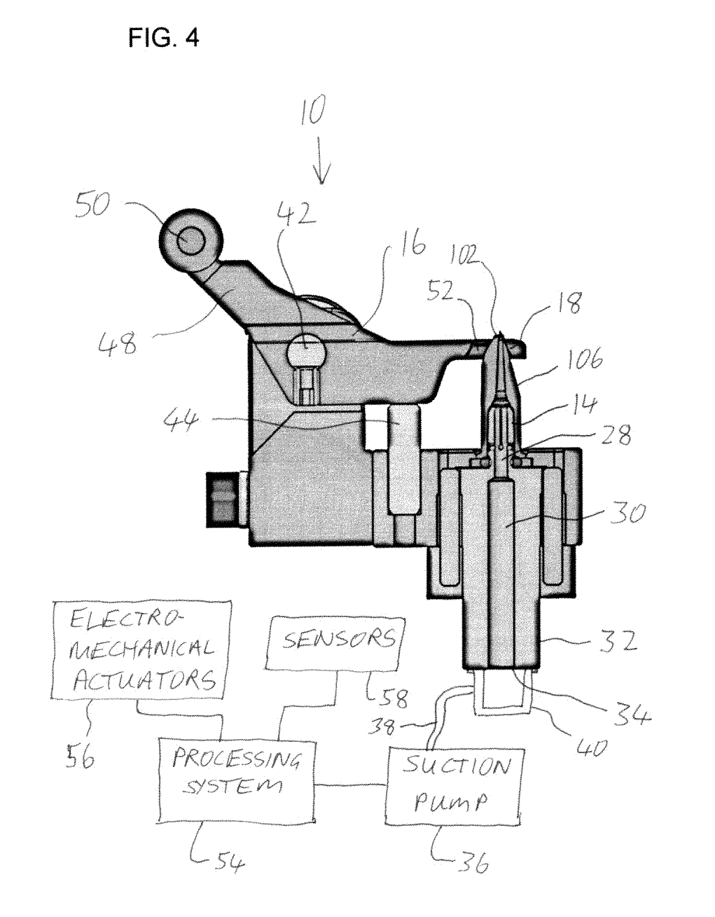

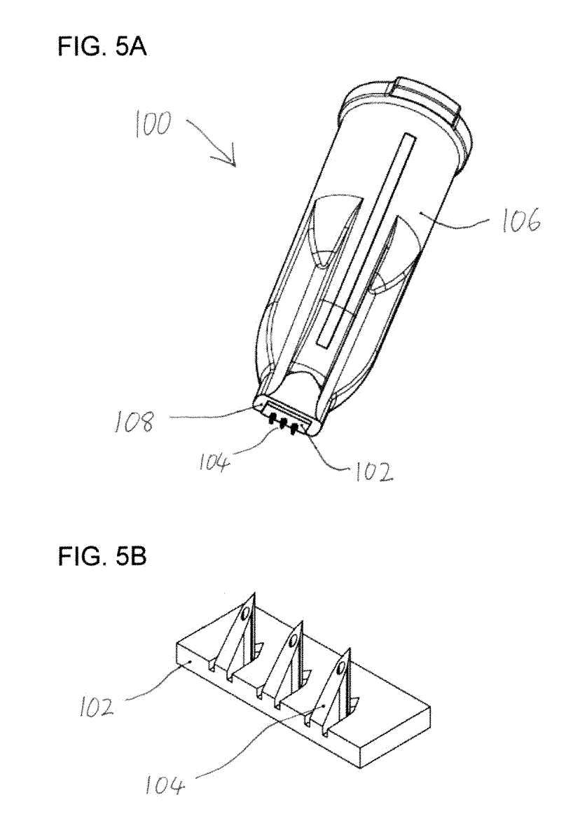

As best seen in FIG. 4, flexible collet 14 is in some particularly preferred implementations an internal collet configured for gripping an internal bore of adapter 106. In the particularly preferred but non-limiting case illustrated here, the internal bore of adapter 106 is a female luer connector suitable, for example, for mating with a standard male luer connector such as is common on standard syringes. A suitable design of collet 14 for gripping such a bore typically employs a plurality of outwardly-resilient elements for engaging the internal surface of the luer connector. According to one option as illustrated here, the collet may be a form similar to a male luer connector formed with a number of radial slots which render the structure radially compressible. It will be understood that collet 14 can readily be adapted to grip internal bores of other types of connector. External collets (not shown) may also be used where more appropriate to the type of connector structure, or other feature of the adapter, to be gripped.

According to another particularly preferred but non-limiting feature, flexible collet 14 has an internal channel 28 that is in sealed interconnection with a vacuum channel 30 passing through base 12. Vacuum channel 30 preferably continues within a downwardly extending column 32 which extends through a work table (not shown) so as to provide a vacuum port 34 accessible from beneath the table. Provision of a fluid flow channel passing through flexible collet 14 to the inside of adapter 106 serves one or more of a number of different functions. Firstly, after placement of the microprotrusion substrate 102 over an aperture in attachment surface 108, suction is preferably applied to vacuum port 34, such as from a suction pump 36 and connecting conduit 38, so as to retain the substrate in the correct position during application of adhesive and bonding. Where a moving work table is used to carry the clamp and the adapter between successive work stations, the vacuum can advantageously be provided to vacuum port 34 via a continuous manifold 40 extending beneath, and in sliding sealing relation to the underside of, the table so that suction is maintained continuously between the station at which the substrate 102 is first placed on the adapter 106 and the station at which initial fixing of the substrate to the adapter is achieved. Additionally, or alternatively, after completion of the attachment, positive pressure applied to port 34 together with pressure monitoring (not shown) can be used to verify that openings of hollow microneedles 104 are not obstructed, and/or to ensure that the chip is sufficiently well attached so as not to be displaced by the positive pressure up to a predefined limit.

In order to ensure sealing interconnection of internal channel 28 to the inside of adapter 106, any slots formed in collet 14 to provide flexibility extend only to a level which is overlapping with adapter 106 when it is positioned on the collet. Optionally, an appropriately positioned resilient sealing element (not shown) may be provided to enhance the seal.

Displaceable fixture 16 is most preferably mounted to base 12 via a hinge 42 which defines its path of motion between its lowered position (FIGS. 1, 3 and 4) at its raised position (FIG. 2). A mechanical stop 44 preferably abuts part of fixture 16 to precisely define the lowered position. In certain particularly preferred embodiments, fixture 16 is biased towards the lowered position by a spring arrangement including at least one spring 46. Displaceable fixture 16 is preferably displaced to its raised position robotically, for example, by downward force applied to a rear lever arm 48, generating a rocking or seesaw motion of displaceable fixture 16 about hinge 42. To facilitate operation by a robotic actuator (not shown), rear lever arm 48 may be provided with a bearing-mounted roller 50.

In the particularly preferred but non-limiting exemplary embodiment illustrated here, locating edges 18 and 20 are implemented as internal surfaces of an aperture 52 passing through displaceable fixture 16. This provides additional protection for the stable alignment of adapter 106, protecting it from inadvertent knocks from other objects in the vicinity of the device. As mentioned earlier, when adapter 106 is positioned on flexible collet 14, attachment surface 108 projects above displaceable fixture 16, making the attachment surface available for attachment of the microprotrusion device.

It should be noted that locating edges 18 and 20 may achieve alignment in one of two ways. In one case, employed here in relation to edges 20, adapter 106 may be centered between a pair of edges which descend either side of the adapter. To facilitate this centering action, an uppermost part of the adapter is preferably formed with a taper (i.e., outwardly sloped surfaces) against which edges 20 descend as the displaceable fixture 16 descends, thereby progressively displacing the top of the adapter laterally from its initial position until it is centered. Edge 18, on the other hand, is in this case an unopposed edge, i.e., where the opposing side of aperture 52 is spaced sufficiently from edge 18 that it doesn't play a part in aligning adapter 106. In this case, it is required that the robotic device for initial placement of adapter 106 onto flexible collet 14 positions adapter 106 with a slight offset from its final desired position so as to ensure that it comes in contact with edge 18 as edge 18 descends. This latter arrangement is particularly preferred where the form of the adapter has significantly outwardly-inclined surfaces in the plane of contact of the locating edges, which would make the separation of a pair of facing edges highly sensitive to the vertical position of the adapter.

FIG. 4 shows schematically a number of additional components of a system employing clamp 10. Specifically, a control system is typically implemented as a processing system 54, including one or more processors, which may be dedicated hardware or general-purpose hardware operating under suitable software and/or firmware, to control operation of various parts of a production system including clamp 10. Thus processing system 54 typically controls suction pump 36 and various actuators 56, which typically include actuator 26 of depressor assembly 22 and a robotic actuator (not shown) for depressing rear lever arm 48. Processing system 54 typically also receives inputs from various other sensors 58, which monitor the states of various other production system components and/or provide information to facilitate quality control of the outcomes of various operations performed by the system. Clamp 10 is preferably part of a production system which includes a plurality of stations, each provided with a clamp 10, with either the clamp advancing from station to station or the other equipment for performing sequential assembly steps moving between fixed stations. In one particularly preferred example, clamps 10 are arrayed around a circular work table which rotates around a central axis to advance the clamps from station to station. Typical but non-limiting examples of stations may include: a robotic placement device for lifting displaceable fixture 16, positioning adapter 106 on flexible collet 14 and lowering fixture 16; depressor arrangement 22; a robotic placement device for the microprotrusion (microneedle) device; an adhesive delivery system; an adhesive curing system; a robotic capping station; and a quality control station.

A particularly preferred sequence for assembling a microprotrusion device according to the teachings of the present invention includes, amongst other steps:
with fixture 16 in its raised position, positioning an adapter 106 on flexible collet 14 so as to be gripped by flexible collet 14;
displacing fixture 16 to its lowered position so that locating edges 18 and 20 abut surfaces of adapter 106 to achieve precise positioning of the adapter in two dimensions;
lowering depressor element 24 into engagement with an upward-facing surface of adapter 106 so as to press the adapter further into engagement with flexible collet 14, thereby bringing the attachment surface 108 of the adapter to a predefined level relative to base 12;
attaching to attachment surface 108 a substrate 102 with a least one microprotrusion 104 projecting therefrom, so as to form the microprotrusion device;
applying suction via a vacuum channel to the internal channel of the flexible collet;
measuring a pressure within the internal channel; and
determining based upon the pressure whether sealing attachment of the substrate to the adapter has been achieved.

As mentioned earlier, the clamp if the present invention is particularly useful in a production process for producing microneedle devices, and in particular, devices employing hollow-microneedle devices for drug delivery and the like. One particularly preferred device is a microneedle device in which the microneedles are integrally formed with the substrate from a single crystal of silicon, and in particular, a device in which the microneedles are asymmetric microneedles bounded by one or more surfaces standing substantially upright relative to the surface of the substrate intersecting with an oblique surface, and wherein a fluid flow bore of the hollow microneedles intersects with the oblique surface. Microneedle components of this type, and various devices employing such components, are commercially available from Nanopass Technologies Ltd. (Israel).

It will be appreciated that the above descriptions are intended only to serve as examples, and that many other embodiments are possible within the scope of the present invention as defined in the appended claims.

What is claimed is:

1. A clamp for use in assembly of a microprotrusion device in which a substrate with a least one microprotrusion is to be attached to an adapter having an elongated body terminating in an attachment surface to which the substrate is to be attached, the clamp comprising:
(a) a base;
(b) a flexible collet mounted on said base so as to project upwards from said base, said flexible collet being configured for repositionably gripping at least one feature of the adapter; and
(c) a displaceable fixture having at least two locating edges, said fixture being displaceably mounted to said base so as to be displaceable between a raised position for positioning an adapter on said flexible collet and a lowered position in which locating edges abut surfaces of the adapter to achieve precise positioning of the adapter in two dimensions, and wherein in said lowered position, the attachment surface of the adapter projects above said locating edges of said fixture so as to be accessible for attachment of said substrate.

2. The clamp of claim 1, wherein said flexible collet is an internal collet configured for gripping an internal bore of the adapter.

3. The claim of claim 2, wherein said flexible collet has an internal channel, and further comprising a vacuum channel passing through said base, said vacuum channel being in sealing interconnection with said flexible collet to allow application of suction via said internal channel of said flexible collet.

4. The clamp of claim 1, wherein said displaceable fixture is mounted to said base via a hinge.

5. The clamp of claim 1, wherein said locating edges are implemented as internal surfaces of an aperture passing through said displaceable fixture.

6. The clamp of claim 1, further comprising a depressor assembly comprising a depressor element configured for engaging an upward-facing surface of the adapter and an actuator associated with said depressor element and configured to selectively lower said depressor element so as to press the adapter further into engagement with said flexible collet, thereby bringing the attachment surface of the adapter to a predefined level relative to said base.

7. The clamp of claim 1, further comprising a spring arrangement deployed so as to bias said displaceable fixture towards said lowered position.

8. A method for assembling a microprotrusion device comprising the steps of:
(a) providing the clamp of claim 1;
(b) with said fixture in said raised position, positioning an adapter on said flexible collet so as to be gripped by said flexible collet, said adapter having an elongated body terminating in an attachment surface;
(c) displacing said fixture to said lowered position so that said locating edges abut surfaces of the adapter to achieve precise positioning of the adapter in two dimensions; and
(d) attaching to said attachment surface a substrate with a least one microprotrusion projecting therefrom to as to form a microprotrusion device.

9. The method of claim 8, further comprising, prior to said attaching, lowering a depressor element into engagement with an upward-facing surface of the adapter so as to press the adapter further into engagement with said flexible collet, thereby bringing the attachment surface of the adapter to a predefined level relative to said base.

10. The method of claim 8, wherein said flexible collet is an internal collet that grips an internal bore of said adapter.

11. The method of claim 10, wherein said flexible collet has an internal channel, the method further comprising applying suction via said internal channel to the internal bore of said adapter so as to temporarily retain said substrate in position on said attachment surface during said attaching.

12. The method of claim 8, wherein said at least one microprotrusion comprises at least one hollow microneedle.

13. The method of claim 8, wherein said at least one microprotrusion is implemented as a plurality of microprotrusions.

14. The method of claim 8, wherein said substrate and said at least one microprotrusion are integrally formed from a single crystal of silicon.

15. The method of claim 8, wherein said adapter is formed with a female luer connector.
